(19) United States
(12) Patent Application Publication
(10) Pub. No.: US 2015/0246185 A1
(43) Pub. Date: Sep. 3, 2015

(54) PREFILLED SYRINGE

(71) Applicants: TRANSCOJECT GMBH, Neumünster (DE); BAYER PHARMA AG, Berlin (DE)

(72) Inventor: Jochen Heinz, Flintbek (DE)

(73) Assignee: TRANSCOJECT GMBH, Neumünster (DE)

(21) Appl. No.: 14/430,363

(22) PCT Filed: Sep. 26, 2013
(86) PCT No.: PCT/EP2013/070140

§ 371 Date: Mar. 23, 2015

Related U.S. Application Data
(60) Provisional application No. 61/706,047, filed on Sep. 26, 2012.

(51) Int. Cl. A61M 5/50; B29C 45/16

(52) U.S. Cl. CPC A61M 5/5086; B29C 45/1676; A61M 2207/00; B29K 2023/12

(57) ABSTRACT

A prefilled syringe includes a syringe cylinder (2), which has an open axial side, closed by a plunger, and another side closed by a Luer lock connection (4), molded on an end wall. A flowable medium (8) fills the syringe cylinder (2), a soft-elastic stopper (9) closes at least the Luer (5), and a tamper-evident closure (15) surrounds the outside of the stopper (9) and which has a predetermined breaking point. The syringe cylinder (2), the end wall (3), and the Luer lock connection (4) are formed as a single piece in the form of a transparent injection-molded plastic part. The tamper-evident closure (15) includes the same or a similar plastic as the syringe cylinder (2) and is welded to the end wall (3) of the syringe cylinder (2). The welding connection forms the predetermined breaking point, and the stopper (9) is integrated into the tamper-evident closure (15).

### CROSS REFERENCE TO RELATED APPLICATIONS

This application is a United States National Phase Application of International Application PCT/EP2013/070140 filed Sep. 26, 2013 and claims the benefit of priority of U.S. Provisional Patent Application 61/706,047 filed Sep. 26, 2012, the entire contents of which are incorporated herein by reference.

### FIELD OF THE INVENTION

The invention relates to a prefilled syringe which comprises a syringe cylinder which at one axial side is designed in an open manner and is closed by a plunger and at the other axial side comprises a Luer lock connection integrally formed on a face wall terminating the syringe cylinder and closed with a plug.

### BACKGROUND OF THE INVENTION

Such prefilled syringes are particularly increasingly applied in medicine and are filled with a flowable medium, i.e. typically a liquid or pasty substance, for example a medication or a contrast means. The ejection of this medium is effected by way of the plunger which is inserted into the syringe cylinder, by which means the medium exits through the opening in the Luer cone of the Luer lock connection. A plug of soft-elastic material which closes at least the Luer within the Luer lock connection is provided, in order to ensure that the medium located in the syringe cylinder does not exit out of the Luer lock connection before its use, and the syringe cylinder is hermetically closed, in particular is protected from the penetration of germs. Thereby, it is particularly with medical products that it is to be ensured that one can immediately recognize externally, as to whether the plug has already been opened once. A tamper-evident closure is provided for this, and this comprises a separating web which must be destroyed, in order to get to the plug, in particular to remove the plug from the Luer.

For syringe cylinders consisting of glass, as is applied almost exclusively with medical products, it is counted as belonging to the state of the art, to stick a temper-evident closure consisting of plastic together with a Luer connection as well as a plug closing this, on the end of the syringe cylinder which is at ejection side, in the manner of a detent connection (EP 0 397 951 A1). The problem with such tamper-evident closures is the fact that these on the one hand must be so elastic that they can be pushed over a bead at the discharge-side end of the syringe cylinder in the manner of a detent, but on the other hand the detent connection however is designed such that the tamper-evident closure breaks at a predefined breaking location on opening, and it is not the detent connection which is detached. This is quite demanding with regard to manufacturing technology and requires a high precision on manufacture.

The tamper-evident closure which is known from DE 102 47 965 A1 and with which the syringe cylinder and the Luer-lock are designed as one piece as a plastic injection molded part, and the plug and tamper-evident closure on the other hand are connected to the syringe cylinder via a material-fit connection, is more favorable inasmuch as this is concerned. The disadvantage with the designs which are shown there is that firstly the plug must be manufactured as a separate component and must be joined into the tamper-evident closure likewise manufactured as a separate component, whereupon the thus formed unit is welded or bonded to the syringe cylinder. After opening the tamper-evident closure, a part of this closure always remains at the syringe side due to the predefined breakage location provided in the tamper-evident closure, and this is disadvantageous for several reasons. Thus, on the one hand, the Luer is not accessible at its outer periphery and on the other hand the destroyed separating web always entails a certain risk of injury. Although it is alternately envisaged there, to design the weld seam between the syringe cylinder and the tamper-evident closure itself as a separating web, it has however been found to be problematic to design the weld seam as a separating web, and a risk of injury also remains there on destroying the separating web.

Finally, the use of plastic for syringe cylinders for prefilled syringes in medical technology has not proven its worth, which is why syringe cylinders manufactured of special glass are always applied with highly pure products.

### SUMMARY OF THE INVENTION

Against this background, it is the object of the present invention, to provide a prefilled syringe which largely avoids the disadvantages mentioned above, is inexpensive in manufacture and is reliable and safe in application.

The prefilled syringe according to the invention comprises a syringe cylinder which at one axial side is designed open and is closed by a plunger, as is common with such syringes, but however at the other axial side comprises a Luer lock connection which is integrally formed on a face wall terminating the syringe cylinder, i.e. is designed as one piece with the syringe cylinder and with the face wall as a plastic injection molded part. The flowable medium which is located in the syringe and which can be pasty, liquid or also gaseous as the case may be, is enclosed peripherally by the syringe cylinder, axially at one side by the plunger, and at the other side by the face wall with the Luer lock connection and the soft-elastic plug. Thereby, the plug sealingly closes at least the Luer, i.e. the actual discharge cone, within the Luer lock connection. The plug is surrounded to the outside by a tamper-evident closure which has a predetermined breakage location. The tamper-evident closure is designed of the same type or equal type of plastic as the syringe cylinder and is welded to the face wall of the syringe cylinder, wherein according to the invention, the weld connection forms the predetermined breakage location, and the plug is integrated into the tamper-evident closure. The contents of the syringe can be seen from the outside, and specifically through the syringe cylinder as well as through the face wall to into the Luer lock connection, since the syringe cylinder is formed from a transparent plastic according to the invention. The tamper-evident closure is preferably but not necessarily likewise formed from a transparent plastic. In any case, it is formed from the same type or similar type of plastic, so that the welding ability between the tamper-evident closure and the face wall of the syringe cylinder is ensured.

A face wall in the context of the invention is to be understood as the whole region between the Luer lock connection and the outer periphery of the syringe cylinder. The tamper-evident closure, as the case may be, can thus be aligned to the syringe cylinder or project beyond this in a complete or partial manner. The welding procedure can be carried out and controlled in a simple manner, even with large-scale manufacture, due to the welding of the tamper-evident closure onto the face wall of the syringe cylinder.

Thereby, the significant advantage of the design according to the invention lies not in providing a separating web as is the case with the state of the art, but in the weld connection itself forming the predetermined breakage location which, in particular in combination with the transparent design of the components, has the great advantage that an almost completely projection-free surface remains on the face wall of the syringe cylinder after the detachment of the tamper-evident closure from the syringe, thus after removing the weld connection or overcoming the weld connection, and this surface neither entails the danger of injury, nor does it restrict the view into the inside of the syringe cylinder. It is precisely the latter which is of great significance in the medical field, in order to rule out gas bubbles being inadvertently entrained, or also in order to merely be able to check the controlled discharge of the medium from the syringe cylinder into the Luer lock connection by way of a viewed control.

The correct seat of the plug and the integrity of the medium located within the syringe cylinder can already be ascertained from the outside before the opening of the tamper-evident closure, in particular if this is likewise designed is a transparent manner, which is advantageous. The solution according to the invention thus in comparison to the state of the art thus represents a considerably less expensive solution that the syringe cylinders which until now have consisted of glass, and at the same time however represents a considerably more reliable solution and one which can be more easily managed, than is known with previous syringes consisting of plastic.

It is particularly advantageous if the plug and the tamper-evident closure are manufactured with the 2K injection molding method. By way of this, it is possible to form the plug from a suitable, soft-elastic, thermoplastic material and the tamper-evident closure from a comparatively hard plastic, similarly to the syringe cylinder. 2K injection molding methods are technically easily manageable and thus represent an inexpensive possibility of manufacturing two components which per se are independent of one another, quasi in a single-piece manner in an injection molding method. Thus, the assembly of the plug within the tamper-evident closure is done away with, by which means the manufacturing costs of the prefilled syringe can be lowered further.

According to one advantageous further development of the invention, the tamper-evident closure and the plug are positively connected to one another. Such a positive fit connection ensures that the plug is arranged within the tamper-evident closure with a positive fit, thus is always handled together with this. Such a fashioning of the component provides the advantage that the materials for the plug and tamper-evident closure and which are used with the 2K injection molding method can be selected with a greater degree of freedom, since these neither need to assume a material-fit connection or any other adhesive connection, but in the extreme case can be connected to one another exclusively by way of a positive-fit. Thereby, the positive fit between the plug and the tamper-evident closure is advantageously formed to the outside and is formed in the rotation direction about the middle longitudinal axis. It is ensured by way of this arrangement that the plug is always co-moved on rotating as well as on tilting the tamper-evident closure on the syringe, and with this, the plug is also reliably co-moved and finally removed on destroying the predetermined breakage location, i.e. the weld connection between the tamper-evident closure and the face wall of the syringe cylinder. Since the tamper-evident closure peripherally surrounds the plug, a comparatively large hand force can be applied onto the plug via this tamper-evident closure, given a suitable positive-fit connection, so that even with a firm seat of the plug within the Luer connection or the Luer lock connection, when the plug also peripherally surrounds the Luer connection, a simple release by hand force is ensured despite this.

The syringe cylinder with the integrally formed Luer lock connection and, as the case may be, also the tamper-evident closure advantageously consists of polyolefin. Numerous combinations are known from this group of plastics and these fulfil special demands on the material for a prefilled syringe, i.e. in particular are formed in a transparent manner, ensure a high dimensional stability on injection molding and provide a large barrier with respect to a penetration of the medium which is stored in the syringe cylinder. An inexpensive plastic for the syringe cylinder and the integrally formed Luer lock connection or the tamper-evident closure is polypropylene. The syringe cylinder can alternatively consist of a cyclo-olefin copolymer or of another suitable thermoplastic material.

The syringe cylinder usefully consists of a barrier plastic, wherein the barrier characteristics are directed to the filling medium.

The plug advantageously consists of a thermoplastic polymer and is somewhat soft and elastic in comparison to the material of the syringe cylinder. A particularly suitable material for this is thermoplastic polyurethane which can be easily managed with injection molding technology, in particular 2K injection molding technology.

The invention is hereinafter explained in more detail by way of one embodiment example represented in the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a greatly simplified, schematic representation, showing the discharge-side end of a prefilled syringe with an applied tamper-evident closure, in a longitudinal section; and

FIG. 2 is a sectional view taken along the section line A-A in FIG. 1.

### DESCRIPTION OF THE PREFERRED EMBODIMENTS

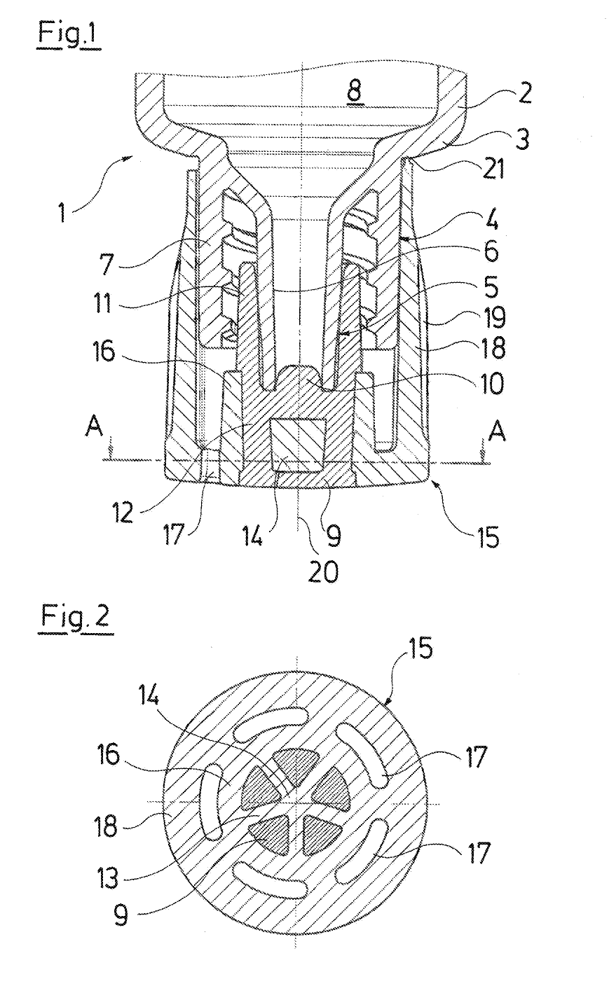

Only the discharge-side part of a prefilled syringe 1 is represented in FIG. 1. This syringe comprises a longitudinally extended syringe cylinder 2 which at its axial end which is not visible in FIG. 1 is designed in an open manner and is closed by way of a plug which is likewise not represented and which is displaceably led within the cylinder 2. The syringe cylinder 2 at the other axial side which is shown in FIG. 1 is closed by a face wall 3, on which a Luer lock connection 4 is integrally formed. The syringe cylinder 2, the face wall 3 and the Luer lock connection 4 are designed as a single-piece plastic injection molded part and consist of a material as has been initially and advantageously specified beforehand. The Luer lock connection 4 in the manner known per se consists of an inner Luer cone 5 which corresponds to a common Luer connection and comprises an inner through-channel 6 to the syringe cylinder 2. The Luer connection 5 is surrounded by a cylindrical section 7 which on its inner side carriers an inner thread and together with the Luer connection 5 forms the Luer lock connection 4 in the manner known per se.

The medium 8 which is located in the syringe cylinder 2 is hermetically closed peripherally by the syringe cylinder 2, at the open axial side which is not visible in FIG. 1 by the plug, and at the other axial side closed off by the face wall 3 and the Luer lock connection 4 by way of a plug 9. The plug 9 consists of a soft-elastic thermoplastic material and comprises a projection 10 which is directed into the through-channel 6 of the Luer connection 5 in a cone-shaped manner and which sealingly closes the through-channel 6 to the outside. An annular section 11 connects to the projection 10 at a radial distance and this section projects significantly beyond the projection 10 to the syringe cylinder 2, in its axial length. This annular section 11 peripherally surrounds the Luer cone 5 and thereby an annular groove into which the free end of the Luer cone 5 engages is formed between the projection 10 and the annular section 11. The sealing is effected firstly by way of the projection 11 which sealingly closes the free end of the through-channel 6, and on the other hand by way of the annular section 11 which sealingly bears on the outer periphery of the Luer cone 5. The projection 10 and the annular section 11 merge into an essentially cylindrical section 12, through which five radially running webs 13 running out into a central space 14 pass.

A plastic component forming a tamper-evident closure 15 passes through the plug 9, in the region of the webs 13 and the centre space 14. This tamper-evident closure 15, apart from the sections projecting into the free spaces 13 and 14 comprises an annular section 16 which surrounds the plug 9 up to close to the axial end of the projection 10. This annular section 16 is connected via a region interrupted by way of annularly arranged recesses 17, to a radially widened, annular section 18 which with its cylindrical inner diameter bears with little play on the outer periphery of the cylindrical section 7 of the Luer lock connection 4 and reaches up to the face wall 3 where it is welded to this in sections. Thereby, the annular section 18 runs in a tapering manner to the face wall 3 and comprises axis-parallel ribs 19 increasing the gripping ability of this section.

As is evident from FIG. 1, the annular section 18 is not welded to the face wall 3 in a completely peripheral manner, but only in sections, similarly to the arrangement of recesses 17 on the other axial side of the tamper-evident closure. Due to the fact that the annular section 18 is welded to the face wall 3 of the syringe cylinder 2 only in sections, a predetermined breakage location is formed between these two components and this can be broken open by way of a simple hand force. The weld connection is thereby released, i.e. the face wall 3 remains almost in its initial from after the opening of the tamper-evident closure, i.e. it remains plane and smooth as well as essentially transparent, so that the view into the inside of the syringe 1 and thus to the filling medium 8 through the transparent plastic of the syringe cylinder is possible. Thus, in particular, the ejection/discharge region of the syringe 1 can be viewed after the removal of the tamper-evident closure 15 and thus can be optically controlled in a simple manner. The surface of the face wall 3 is also smooth and is essentially free from the remains of the tamper-evident closure 15.

The plug 9 which in the represented embodiment is not designed in a transparent manner, is passed through by the tamper-evident closure 15 which likewise consist of a transparent thermoplastic plastic, so that one the one hand it can be ensured that after the assembly of this component which consists of the plug 9 and the tamper-evident closure 15 and which is manufactured with the 2K injection molding method, the correct seat of the plug 9 is always ensured on the one hand, and that a movement and thus removal of the plug 9 when opening the weld connection is ensured on the other hand. It is evident that the materials forming the plug 9 and the tamper-evident closure 15 do not need to assume any type of material-fit connection with one another, but that the positive fit between these components and which is inherent of the design is sufficient to connect these to one another in a fixed and non-detachable manner.

The component of the plug 9 and tamper-evident closure 15 which is manufactured with the 2K injection molding method is designed such that the inner side of the annular section 18 is led on the outer side of the cylindrical section 7 of the Luer lock connection 4, on applying this component onto the syringe, as is illustrated in the sectioned representation according to FIG. 1. Firstly, the annular section 18 is applied onto the Luer connection 5 by way of this cylindrical guiding, wherein the projection 10 is sealingly integrated into the end of the through-channel 6 in the end phase of the assembly procedure. It is ensured by way of this guiding that the plug 9 is always closes the Luer connection 5 in the designated manner. These weld seam sections can also be created in a simple manner with large-scale manufacture due to the arrangement of the ring sections 21, with which the tamper evident closure 15 is welded to the face wall 3 of the syringe, since the regions are easily accessible. This manufacturing process can not only be monitored by viewed control, but also by way of suitable other testing methods. Finally, at this exposed location of the syringe, one can immediately recognize if the tamper-evident closure 15 has been broken open, i.e. the weld connection is detached from the face wall 3. This is particularly easily visible due to the fact that the welding is not effected in a continuous peripheral manner, but only in the region of the face side sections 21, so that typically a rotation about the middle longitudinal axis 20 is effected on removing and reattaching the tamper-evident closure 15 and this rotation is visible at the face wall 3, since then the weld locations are then no longer aligned with the face-side sections 21.

While specific embodiments of the invention have been shown and described in detail to illustrate the application of the principles of the invention, it will be understood that the invention may be embodied otherwise without departing from such principles.

1. A prefilled syringe comprising:
a plunger;
a syringe cylinder with one axial side with an opening closed by the plunger, and which comprises a Luer lock connection comprising a Luer at another axial side, said Luer lock connection being integrally formed on a face wall of the syringe cylinder, closing off the syringe cylinder;
a flowable medium filled into the syringe cylinder;
a soft-elastic plug which closes at least the Luer and with
a tamper-evident closure which at least partially surrounds the plug to an outside of the plug and has a predetermined breakage location, wherein the syringe cylinder, the face wall and the Luer lock connection are formed as one piece as a transparent plastic injection molded part, the tamper-evident closure comprising a same type or similar type of plastic as the syringe cylinder and being welded to the face wall of the syringe cylinder via a weld connection, wherein the weld connection forms the predetermined breakage location and the plug is integrated into the tamper-evident closure.

2. A prefilled syringe according to claim 1, wherein the plug and the tamper-evident closure are manufactured with the 2K injection molding method.

3. A prefilled syringe according to claim 1, wherein the tamper-evident closure and the plug are positively connected to one another.

4. A prefilled syringe according to claim 1, wherein the tamper-evident closure is designed in a transparent manner.

5. A prefilled syringe according to claim 1, wherein the positive-fit between the plug and the tamper-evident closure is formed to the outside and about the middle longitudinal axis in the rotation direction.

6. A prefilled syringe according to claim 1, wherein the syringe cylinder consist of polyolefin.

7. A prefilled syringe according to claim 5, wherein the syringe cylinder is formed of polypropylene.

8. A prefilled syringe according to claim 1, wherein the syringe cylinder is formed of a cyclo-olefin copolymer.

9. A prefilled syringe according to claim 1, wherein the syringe cylinder is formed of a barrier plastic, whose barrier characteristics are matched to the filling medium.

10. A prefilled syringe according to claim 1, wherein the plug is formed of a thermoplastic polymer.

11. A prefilled syringe according to claim 1, wherein the plug is formed of a thermoplastic polyurethane.
